(19) United States
(12) Patent Application Publication
(10) Pub. No.: US 2019/0381034 A1
(43) Pub. Date: Dec. 19, 2019

(54) PHARMACEUTICAL COMPOSITION AND METHOD FOR ACUTE ON CHRONIC LIVER FAILURE AND RELATED LIVER DISEASES

(71) Applicant: Ming Fang, Walnut Creek, CA (US)

(72) Inventor: Ming Fang, Walnut Creek, CA (US)

(21) Appl. No.: 16/028,228

(22) Filed: Jul. 5, 2018

Related U.S. Application Data
(60) Provisional application No. 62/685,041, filed on Jun. 14, 2018.

(51) Int. Cl. A61K 31/496; A61K 9/00; A61K 9/48; A61P 1/16

(52) U.S. Cl. CPC A61K 31/496; A61K 9/0019; A61K 9/0053; A61K 47/50; A61P 1/16; A61K 2300/00; A61K 9/4808

(57) ABSTRACT

The present invention provides a composition and method for treating acute on chronical liver failure or a related disease. The method comprises administering to a subject a composition comprising trimetazidine hydrochloride (1-[2,3,4-trimethoxybenzyl] piperazine dihydrochloride) (Trimetazidine or TMZ) or a pharmaceutically acceptable salt thereof according to a dosage regimen.

### CROSS-REFERENCE TO RELATED APPLICATION

This application claims the benefit of U.S. provisional application No. 62/685,041, entitled “PHARMACEUTICAL COMPOSITION AND METHOD FOR ACUTE ON CHRONIC LIVER FAILURE AND RELATED LIVER DISEASES,” filed Jun. 14, 2018, the teaching of which is incorporated herein in its entirety by reference.

### FIELD OF THE INVENTION

The present invention generally relates to a pharmaceutical composition for acute on chronic liver failure and related liver diseases.

### BACKGROUND OF THE INVENTION

Acute on chronic liver failure is a common medical condition and is currently treated using liver-protection drugs. Liver-protection drug refers to the category of drugs used to protect liver functions, characterized in that it is capable of maintaining liver functions, reducing hepatocyte injury, promoting repair and regeneration of injured hepatocyte, and enhancing the detoxifying function of the liver. At present, there is a shortage of large scale evidence-based medicine and action target researches on hepatoprotective drugs. It is known that current hepatoprotective drugs mainly include the detoxifying category (reductive glutathione, tiopronin, etc.), the hepatocyte regeneration promoting category (hepatocyte growth-promoting factor, Polyene Phosphatidylcholine), the energy metabolism promoting category (water-soluble vitamins, coenzymes, potassium aspartate magnesium, ornithine aspartate), the cholagogue category (ademetionine, methionine and Vitamin B1, anethol trithione), and the anti-inflammatory traditional Chinese medicine (silibin, compound glycyrrizin preparation). The effect of any hepatocyte protective drug is relative, and most of the drugs, including most hepatocyte protective drugs, are metabolized by liver, which also increases the liver's load of detoxification and metabolism to some extent.

There are a great variety of existing hepatoprotective drugs, but all with single or unidentified sites of action and relatively limited “hepatoprotective” effects. Since most drugs, not excluding most hepatoprotective drugs, are metabolized by liver, it increases the burden of the liver, which is remarkable in patients suffering from liver failure. Most patients suffering from chronic liver diseases and some suffering from acute liver diseases die of the end stage liver failure. For example, all liver diseases caused by virus, drugs, alcohol, non-alcoholic fatty liver or autoimmunity or genetic and metabolic liver diseases can result in liver failure. In recent years, liver failure is treated mainly through combined treatment, by preventing and treating all sorts of clinical complications, delaying hepatocyte injury and waiting for hepatocyte regeneration, but there is no effective drug to treat the liver failure induced by various liver diseases.

Trimetazidine hydrochloride (1-[2,3,4-trimethoxybenzyl] piperazine dihydrochloride) (TMZ) is described by Yu in PCT/CN2016/080219 for use in acute liver failure, which is uncommon. However, Yu fails to teach or disclose using TMZ for acute on chronic liver failure or related diseases.

Therefore, it is an objective of the present invention to provide compositions and methods for acute on chronic liver failure and related diseases.

Embodiments described below address the above-identified needs and objectives

### SUMMARY OF INVENTION

In one aspect of the present invention, it is provided a method of treating acute on chronical liver failure (ACLF) or a related disease in a subject in need thereof, comprising administering to the subject a composition comprising an effective amount of trimetazidine hydrochloride (1-[2,3,4-trimethoxybenzyl] piperazine dihydrochloride) (Trimetazidine or TMZ) or a pharmaceutically acceptable salt thereof according to a dosage regimen.

In some embodiments of the invention method, optionally in combination with any or all of the various embodiments described herein, the effective amount of TMZ is administered to the subject according to a dosage regimen that comprises a dosage of 20-500 mg every 4-12 hours.

In some embodiments of the invention method, optionally in combination with any or all of the various embodiments described herein, the dosage regimen comprises a dosage of 20-500 mg every 4-12 hours for a course from 1 day to 2 weeks.

In some embodiments of the invention method, optionally in combination with any or all of the various embodiments described herein, the dosage regimen comprises a dosage of 20-500 mg every 4-12 hours for a course from 1 day to 1 month.

In some embodiments of the invention method, optionally in combination with any or all of the various embodiments described herein, the administering comprises oral administration.

In some embodiments of the invention method, optionally in combination with any or all of the various embodiments described herein, the administering comprises parenteral injection.

In some embodiments of the invention method, optionally in combination with any or all of the various embodiments described herein, the administering comprises IV administration.

In some embodiments of the invention method, optionally in combination with any or all of the various embodiments described herein, the composition further comprises a pharmaceutically acceptable carrier.

In some embodiments of the invention method, optionally in combination with any or all of the various embodiments described herein, the pharmaceutical composition reduces activation of resting T lymphocytes to activated lymphocytes in the subject.

In some embodiments of the invention method, optionally in combination with any or all of the various embodiments described herein, the pharmaceutical composition reduces fatty acid metabolism in hepatocytes and promotes aerobic oxidation of glucose in the subject.

In some embodiments of the invention method, optionally in combination with any or all of the various embodiments described herein, the pharmaceutical composition reduces beta-oxidation in hepatocytes and reduces formation of ROS in the subject.

In some embodiments of the invention method, optionally in combination with any or all of the various embodiments described herein, the pharmaceutical composition reduces blood alanine aminotransferase (ALT) level in the subject.

In some embodiments of the invention method, optionally in combination with any or all of the various embodiments described herein, the pharmaceutical composition reduces blood aspartate transaminase (AST) level in the subject.

In some embodiments of the invention method, optionally in combination with any or all of the various embodiments described herein, the related disorder does not include angina pectoris, coronary insufficiency, previous myocardial infarction, coronary heart disease, vertigo or tinnitus.

In some embodiments of the invention method, optionally in combination with any or all of the various embodiments described herein, the related disorder is selected from the group consisting of hepatopathy (liver damage without major inflammation), hepatitis (with inflammation) and cirrhosis (structure damage may or may not have liver failure).

In some embodiments of the invention method, optionally in combination with any or all of the various embodiments described herein, the related disorder is selected from the group consisting of viral hepatitis, non-specific virus hepatitis, drug or medication induced liver injury or damage, toxin induced hepatitis such as herbs or mushroom induced hepatitis or hepatopathy, alcoholic hepatitis, autoimmune hepatitis, alcoholic fatty liver disease, or non-alcoholic fatty liver disease(NAFLD), non-alcoholic steatohepatitis (NASH), metabolic or genetically related liver diseases (Hemochromatosis, Wilson's diseases), ischemic liver injury (shock liver), sepsis (bacteria or other microorganism infection) induced liver failure, and congestive hepatopathy (heart failure induced liver failure).

In some embodiments of the invention method, optionally in combination with any or all of the various embodiments described herein, the subject is a human being or an animal.

In some embodiments of the invention method, optionally in combination with any or all of the various embodiments described herein, the composition comprises a second agent.

In another aspect of the present invention, it is provided a formulation comprising trimetazidine hydrochloride (1-[2,3,4-trimethoxybenzyl] piperazine dihydrochloride) (Trimetazidine or TMZ) or a pharmaceutically acceptable salt thereof and a carrier, the formulation provides a release profile of TMZ or a pharmaceutically acceptable salt thereof, which profile comprising a burst release of the TMZ or a pharmaceutically acceptable salt thereof followed by a sustained release of TMZ or a pharmaceutically acceptable salt thereof.

In some embodiments of the invention formulation, optionally in combination with any or all of the various embodiments described herein, the burst release releases 30 mg to 180 mg of TMZ or a pharmaceutically acceptable salt thereof following administration of the TMZ or a pharmaceutically acceptable salt thereof to a patient.

In some embodiments of the invention formulation, optionally in combination with any or all of the various embodiments described herein, the sustained release releases 10 mg to 30 mg of TMZ or a pharmaceutically acceptable salt thereof every 4 to 6 hours over a period from one day to one month.

In some embodiments of the invention formulation, optionally in combination with any or all of the various embodiments described herein, the sustained release of TMZ or a pharmaceutically acceptable salt thereof is provided by embedding the TMZ or the pharmaceutically acceptable salt thereof in a biocompatible polymeric material, admixing TMZ or the pharmaceutically acceptable salt thereof with a biocompatible polymeric material, or encapsulating or microencapsulating TMZ or the pharmaceutically acceptable salt thereof with a polymeric material.

In some embodiments of the invention formulation, optionally in combination with any or all of the various embodiments described herein, the formulation is an oral formulation or an injection formulation.

In some embodiments of the invention formulation, optionally in combination with any or all of the various embodiments described herein, the formulation comprises any composition disclosed herein. Such composition can be, for example, a composition as described in any of preceding paragraphs [0009]-[00025].

In some other embodiments of the invention formulation, optionally in combination with any or all of the various embodiments described herein, the formulation can be made to specifically exclude a composition of any embodiment disclosed herein. Such composition can be, for example, a composition as described in any of preceding paragraphs [0009]-[00025].

In another aspect of the present invention, it is provided a method of making a formulation, which method comprising

providing trimetazidine hydrochloride (1-[2,3,4-trimethoxybenzyl] piperazine dihydrochloride) (Trimetazidine or TMZ) or a pharmaceutically acceptable salt thereof;
providing a carrier; and
forming the formulation,
wherein the formulation provides a release profile of TMZ or a pharmaceutically accepttable salt thereof, which profile comprising a burst release of the TMZ or a pharmaceutically acceptable salt thereof followed by a sustained release of TMZ or a pharmaceutically acceptable salt thereof.

In some embodiments of the invention method, optionally in combination with any or all of the various embodiments described herein, the burst release releases 30 mg to 180 mg of TMZ or a pharmaceutically acceptable salt thereof following administration of the TMZ or a pharmaceutically acceptable salt thereof to a patient.

In some embodiments of the invention method, optionally in combination with any or all of the various embodiments described herein, the sustained release releases 10 mg to 30 mg of TMZ or a pharmaceutically acceptable salt thereof every 4 to 6 hours over a period from one day to one month.

In some embodiments of the invention method, optionally in combination with any or all of the various embodiments described herein, the sustained release of TMZ or a pharmaceutically acceptable salt thereof is provided by embedding the TMZ or the pharmaceutically acceptable salt thereof in a biocompatible polymeric material, admixing TMZ or the pharmaceutically acceptable salt thereof with a biocompatible polymeric material, or encapsulating or microencapsulating TMZ or the pharmaceutically acceptable salt thereof with a polymeric material.

In some embodiments of the invention method, optionally in combination with any or all of the various embodiments described herein, the formulation is an oral formulation.

In some embodiments of the invention method, optionally in combination with any or all of the various embodiments described herein, the formulation is an injection formulation.

In some embodiments of the invention method, optionally in combination with any or all of the various embodiments described herein, the formulation comprises any composition disclosed herein. Such composition can be, for example, a composition as described in any of preceding paragraphs [0009]-[00025].

In some other embodiments of the invention method, optionally in combination with any or all of the various embodiments described herein, the formulation can be made to specifically exclude a composition of any embodiment disclosed herein. Such composition can be, for example, a composition as described in any of preceding paragraphs [0009]-[00025].

In a further aspect of the present invention, it is provided a method of treating a liver disorder or a related disease, the method comprising administering to a patient in need thereof a formulation disclosed herein, the formulation comprising trimetazidine hydrochloride (1-[2,3,4-trimethoxybenzyl] piperazine dihydrochloride) (Trimetazidine or TMZ) or a pharmaceutically acceptable salt thereof and a carrier, the formulation provides a release profile of TMZ or a pharmaceutically acceptable salt thereof, which profile comprising a burst release of the TMZ or a pharmaceutically acceptable salt thereof followed by a sustained release of TMZ or a pharmaceutically acceptable salt thereof.

In some embodiments of the invention method, optionally in combination with any or all of the various embodiments described herein, the burst release releases 30 mg to 180 mg of TMZ or a pharmaceutically acceptable salt thereof following administration of the TMZ or a pharmaceutically acceptable salt thereof to a patient.

In some embodiments of the invention method, optionally in combination with any or all of the various embodiments described herein, the sustained release releases 10 mg to 30 mg of TMZ or a pharmaceutically acceptable salt thereof every 4 to 6 hours over a period from one day to one month.

In some embodiments of the invention method, optionally in combination with any or all of the various embodiments described herein, the sustained release of TMZ or a pharmaceutically acceptable salt thereof is provided by embedding the TMZ or the pharmaceutically acceptable salt thereof in a biocompatible polymeric material, admixing TMZ or the pharmaceutically acceptable salt thereof with a biocompatible polymeric material, or encapsulating or microencapsulating TMZ or the pharmaceutically acceptable salt thereof with a polymeric material.

In some embodiments of the invention method, optionally in combination with any or all of the various embodiments described herein, the formulation is an oral formulation.

In some embodiments of the invention method, optionally in combination with any or all of the various embodiments described herein, the formulation is an injection formulation.

In some embodiments of the invention method, optionally in combination with any or all of the various embodiments described herein, the liver disorder is acute on chronic liver failure.

In some embodiments of the invention method, optionally in combination with any or all of the various embodiments described herein, the related disorder is selected from the group consisting of he related disorder is selected from the group consisting of viral hepatitis, non-specific virus hepatitis, drug or medication induced liver injury or damage, toxin induced hepatitis such as herbs or mushroom induced hepatitis or hepatopathy, alcoholic hepatitis, autoimmune hepatitis, alcoholic fatty liver disease, or non-alcoholic fatty liver disease(NAFLD), non-alcoholic steatohepatitis (NASH), metabolic or genetically related liver diseases (Hemochromatosis, Wilson's diseases), ischemic liver injury (shock liver), sepsis (bacteria or other microorganism infection) induced liver failure, and congestive hepatopathy (heart failure induced liver failure).

In some embodiments of the invention method, optionally in combination with any or all of the various embodiments described herein, the formulation comprises any composition disclosed herein. Such composition can be, for example, a composition as described in any of preceding paragraphs [0009]-[00025].

In some other embodiments of the invention method, optionally in combination with any or all of the various embodiments described herein, the formulation can be made to specifically exclude a composition of any embodiment disclosed herein. Such composition can be, for example, a composition as described in any of preceding paragraphs [0009]-[00025].

### DETAILED DESCRIPTION OF THE INVENTION

In one aspect of the present invention, it is provided a method of treating acute on chronical liver failure (ACLF) or a related disease in a subject in need thereof, comprising administering to the subject a composition comprising an effective amount of trimetazidine hydrochloride (1-[2,3,4-trimethoxybenzyl] piperazine dihydrochloride) (Trimetazidine or TMZ) or a pharmaceutically acceptable salt thereof according to a dosage regimen.

In some embodiments of the invention method, optionally in combination with any or all of the various embodiments described herein, the effective amount of TMZ is administered to the subject according to a dosage regimen that comprises a dosage of 20-500 mg every 4-12 hours.

In some embodiments of the invention method, optionally in combination with any or all of the various embodiments described herein, the dosage regimen comprises a dosage of 20-500 mg every 4-12 hours for a course from 1 day to 2 weeks.

In some embodiments of the invention method, optionally in combination with any or all of the various embodiments described herein, the dosage regimen comprises a dosage of 20-500 mg every 4-12 hours for a course from 1 day to 1 month.

In some embodiments of the invention method, optionally in combination with any or all of the various embodiments described herein, the administering comprises oral administration.

In some embodiments of the invention method, optionally in combination with any or all of the various embodiments described herein, the administering comprises parenteral injection.

In some embodiments of the invention method, optionally in combination with any or all of the various embodiments described herein, the administering comprises IV administration.

In some embodiments of the invention method, optionally in combination with any or all of the various embodiments described herein, the composition further comprises a pharmaceutically acceptable carrier.

In some embodiments of the invention method, optionally in combination with any or all of the various embodiments described herein, the pharmaceutical composition reduces activation of resting T lymphocytes to activated lymphocytes in the subject.

In some embodiments of the invention method, optionally in combination with any or all of the various embodiments described herein, the pharmaceutical composition reduces fatty acid metabolism in hepatocytes and promotes aerobic oxidation of glucose in the subject.

In some embodiments of the invention method, optionally in combination with any or all of the various embodiments described herein, the pharmaceutical composition reduces beta-oxidation in hepatocytes and reduces formation of ROS in the subject.

In some embodiments of the invention method, optionally in combination with any or all of the various embodiments described herein, the pharmaceutical composition reduces blood alanine aminotransferase (ALT) level in the subject.

In some embodiments of the invention method, optionally in combination with any or all of the various embodiments described herein, the pharmaceutical composition reduces blood aspartate transaminase (AST) level in the subject.

In some embodiments of the invention method, optionally in combination with any or all of the various embodiments described herein, the related disorder does not include angina pectoris, coronary insufficiency, previous myocardial infarction, coronary heart disease, vertigo or tinnitus.

In some embodiments of the invention method, optionally in combination with any or all of the various embodiments described herein, the related disorder is selected from the group consisting of hepatopathy (liver damage without major inflammation), hepatitis (with inflammation) and cirrhosis (structure damage may or may not have liver failure).

In some embodiments of the invention method, optionally in combination with any or all of the various embodiments described herein, the related disorder is selected from the group consisting of viral hepatitis, non-specific virus hepatitis, drug or medication induced liver injury or damage, toxin induced hepatitis such as herbs or mushroom induced hepatitis or hepatopathy, alcoholic hepatitis, autoimmune hepatitis, alcoholic fatty liver disease, or non-alcoholic fatty liver disease(NAFLD), non-alcoholic steatohepatitis (NASH), metabolic or genetically related liver diseases (Hemochromatosis, Wilson's diseases), ischemic liver injury (shock liver), sepsis (bacteria or other microorganism infection) induced liver failure, and congestive hepatopathy (heart failure induced liver failure).

In some embodiments of the invention method, optionally in combination with any or all of the various embodiments described herein, the subject is a human being or an animal.

In some embodiments of the invention method, optionally in combination with any or all of the various embodiments described herein, the composition comprises a second agent.

In another aspect of the present invention, it is provided a formulation comprising trimetazidine hydrochloride (1-[2,3,4-trimethoxybenzyl] piperazine dihydrochloride) (Trimetazidine or TMZ) or a pharmaceutically acceptable salt thereof and a carrier, the formulation provides a release profile of TMZ or a pharmaceutically acceptable salt thereof, which profile comprising a burst release of the TMZ or a pharmaceutically acceptable salt thereof followed by a sustained release of TMZ or a pharmaceutically acceptable salt thereof.

In some embodiments of the invention formulation, optionally in combination with any or all of the various embodiments described herein, the burst release releases 30 mg to 180 mg of TMZ or a pharmaceutically acceptable salt thereof following administration of the TMZ or a pharmaceutically acceptable salt thereof to a patient.

In some embodiments of the invention formulation, optionally in combination with any or all of the various embodiments described herein, the sustained release releases 10 mg to 30 mg of TMZ or a pharmaceutically acceptable salt thereof every 4 to 6 hours over a period from one day to one month.

In some embodiments of the invention formulation, optionally in combination with any or all of the various embodiments described herein, the sustained release of TMZ or a pharmaceutically acceptable salt thereof is provided by embedding the TMZ or the pharmaceutically acceptable salt thereof in a biocompatible polymeric material, admixing TMZ or the pharmaceutically acceptable salt thereof with a biocompatible polymeric material, or encapsulating or microencapsulating TMZ or the pharmaceutically acceptable salt thereof with a polymeric material.

In some embodiments of the invention formulation, optionally in combination with any or all of the various embodiments described herein, the formulation is an oral formulation or an injection formulation.

In some embodiments of the invention formulation, optionally in combination with any or all of the various embodiments described herein, the formulation comprises any composition disclosed herein. Such composition can be, for example, a composition as described in any of preceding paragraphs [00052]-[00068].

In some other embodiments of the invention formulation, optionally in combination with any or all of the various embodiments described herein, the formulation can be made to specifically exclude a composition of any embodiment disclosed herein. Such composition can be, for example, a composition as described in any of preceding paragraphs [00052]-[00068].

In another aspect of the present invention, it is provided a method of making a formulation, which method comprising

providing trimetazidine hydrochloride (1-[2,3,4-trimethoxybenzyl] piperazine dihydrochloride) (Trimetazidine or TMZ) or a pharmaceutically acceptable salt thereof;
providing a carrier; and
forming the formulation,
wherein the formulation provides a release profile of TMZ or a pharmaceutically acceptable salt thereof, which profile comprising a burst release of the TMZ or a pharmaceutically acceptable salt thereof followed by a sustained release of TMZ or a pharmaceutically acceptable salt thereof.

In some embodiments of the invention method, optionally in combination with any or all of the various embodiments described herein, the burst release releases 30 mg to 180 mg of TMZ or a pharmaceutically acceptable salt thereof following administration of the TMZ or a pharmaceutically acceptable salt thereof to a patient.

In some embodiments of the invention method, optionally in combination with any or all of the various embodiments described herein, the sustained release releases 10 mg to 30 mg of TMZ or a pharmaceutically acceptable salt thereof every 4 to 6 hours over a period from one day to one month.

In some embodiments of the invention method, optionally in combination with any or all of the various embodiments described herein, the sustained release of TMZ or a pharmaceutically acceptable salt thereof is provided by embedding the TMZ or the pharmaceutically acceptable salt thereof in a biocompatible polymeric material, admixing TMZ or the pharmaceutically acceptable salt thereof with a biocompatible polymeric material, or encapsulating or microencapsulating TMZ or the pharmaceutically acceptable salt thereof with a polymeric material.

In some embodiments of the invention method, optionally in combination with any or all of the various embodiments described herein, the formulation is an oral formulation.

In some embodiments of the invention method, optionally in combination with any or all of the various embodiments described herein, the formulation is an injection formulation.

In some embodiments of the invention method, optionally in combination with any or all of the various embodiments described herein, the formulation comprises any composition disclosed herein. Such composition can be, for example, a composition as described in any of preceding paragraphs [00052]-[00068].

In some other embodiments of the invention method, optionally in combination with any or all of the various embodiments described herein, the formulation can be made to specifically exclude a composition of any embodiment disclosed herein. Such composition can be, for example, a composition as described in any of preceding paragraphs [00052]-[00068].

In a further aspect of the present invention, it is provided a method of treating a liver disorder or a related disease, the method comprising administering to a patient in need thereof a formulation disclosed herein, the formulation comprising trimetazidine hydrochloride (1-[2,3,4-trimethoxybenzyl] piperazine dihydrochloride) (Trimetazidine or TMZ) or a pharmaceutically acceptable salt thereof and a carrier, the formulation provides a release profile of TMZ or a pharmaceutically acceptable salt thereof, which profile comprising a burst release of the TMZ or a pharmaceutically acceptable salt thereof followed by a sustained release of TMZ or a pharmaceutically acceptable salt thereof.

In some embodiments of the invention method, optionally in combination with any or all of the various embodiments described herein, the burst release releases 30 mg to 180 mg of TMZ or a pharmaceutically acceptable salt thereof following administration of the TMZ or a pharmaceutically acceptable salt thereof to a patient.

In some embodiments of the invention method, optionally in combination with any or all of the various embodiments described herein, the sustained release releases 10 mg to 30 mg of TMZ or a pharmaceutically acceptable salt thereof every 4 to 6 hours over a period from one day to one month.

In some embodiments of the invention method, optionally in combination with any or all of the various embodiments described herein, the sustained release of TMZ or a pharmaceutically acceptable salt thereof is provided by embedding the TMZ or the pharmaceutically acceptable salt thereof in a biocompatible polymeric material, admixing TMZ or the pharmaceutically acceptable salt thereof with a biocompatible polymeric material, or encapsulating or microencapsulating TMZ or the pharmaceutically acceptable salt thereof with a polymeric material.

In some embodiments of the invention method, optionally in combination with any or all of the various embodiments described herein, the formulation is an oral formulation.

In some embodiments of the invention method, optionally in combination with any or all of the various embodiments described herein, the formulation is an injection formulation.

In some embodiments of the invention method, optionally in combination with any or all of the various embodiments described herein, the liver disorder is acute on chronic liver failure.

In some embodiments of the invention method, optionally in combination with any or all of the various embodiments described herein, the related disorder is selected from the group consisting of he related disorder is selected from the group consisting of viral hepatitis, non-specific virus hepatitis, drug or medication induced liver injury or damage, toxin induced hepatitis such as herbs or mushroom induced hepatitis or hepatopathy, alcoholic hepatitis, autoimmune hepatitis, alcoholic fatty liver disease, or non-alcoholic fatty liver disease(NAFLD), non-alcoholic steatohepatitis (NASH), metabolic or genetically related liver diseases (Hemochromatosis, Wilson's diseases), ischemic liver injury (shock liver), sepsis (bacteria or other microorganism infection) induced liver failure, and congestive hepatopathy (heart failure induced liver failure).

In some embodiments of the invention method, optionally in combination with any or all of the various embodiments described herein, the formulation comprises any composition disclosed herein. Such composition can be, for example, a composition as described in any of preceding paragraphs [0009]-[00025].

In some other embodiments of the invention method, optionally in combination with any or all of the various embodiments described herein, the formulation can be made to specifically exclude a composition of any embodiment disclosed herein. Such composition can be, for example, a composition as described in any of preceding paragraphs [0009]-[00025].

### Definitions

Trimetazidine and acute on chronic liver failure

Trimetazidine, characterized in a new use as a hepatoprotective drug in prevention and treatment of liver failure.

Trimetazidine as a hepatoprotective drug as described herein, wherein trimetazidine is capable of: inhibiting activation of resting T lymphocytes to activated lymphocytes and macrophage, reducing release of cytokines, and reducing hepatocyte injury caused by immune system; inhibiting fatty acid metabolism in hepatocytes and optimizing energy metabolism process in hepatocytes; maintaining normal function of mitochondrial permeability transition pores and reducing hepatocyte apoptosis.

Trimetazidine as a hepatoprotective drug as described herein, wherein trimetazidine is capable of: inhibiting fatty acid metabolism, in particular inhibiting mitochondrial ketoacyl thiolase activity, thereby inhibiting cell fatty acid .beta.-oxidation and reducing ROS reaction; wherein trimetazidine is capable of enhancing mitochondrial pyruvate dehydrogenase activity, thereby promoting aerobic oxidation of glucose which, in ischemic cells, requires lower oxygen consumption than .beta.-oxidation to obtain energy, and wherein trimetazidine is capable of optimizing the energy process in hepatocytes, thereby maintaining proper energy metabolism during ischemia.

Trimetazidine as a hepatoprotective drug as described herein, wherein trimetazidine achieves hepatoprotective effects primarily through the following three aspects:

(1) As lymphocyte activation requires ATP provided by fatty acid metabolism, trimetazidine is capable of inhibiting free fatty acid (FFA) metabolism, inhibiting activation of resting T lymphocytes to activated lymphocytes, thereby reducing release of cytokines by activated cytotoxic T lymphocytes, reducing exogenous cell necrosis or apoptosis, and reducing hepatocyte injury caused by human immune system.
(2) As hepatocytes during liver failure are under an adverse environment with high ammonia, low oxygen and high bilirubin, have severely affected cellular generation of ATP, which in turn inhibits development and growth of the hepatocytes, trimetazidine is capable of inhibiting fatty acid metabolism in the hepatocytes, promoting glucose glycolysis and subsequent TCA (tricarboxylicacidcycle) cycle, reducing oxygen atoms required for generating the same amount of ATP in hepatocytes, and facilitating the use of limited oxygen atoms for oxidizing glucose to provide ATP needed by hepatocytes to synthesize active substances comprising RNA, DNA, and protein; wherein it is common in liver injury, especially during liver failure, that the formation of microthrombus in injured liver leads to a hypoxic state for hepatocytes; wherein trimetazidine is also capable of inhibiting FFA (free fatty acid) metabolism, reducing the formation of ROS in hepatocytes, and reducing secondary hepatocyte injury.
(3) As hepatocyte injury causes damage to mitochondria, reduces ATP synthesis and natural immunity, which can be characterized by opening of MPT and efflux of K+ and Ca2+, thereby initiating endogenous apoptosis, as particularly shown in hepatocyte injury caused by high blood ammonia, trimetazidine is capable of maintaining proper release of calcium ions by MPT pores and reducing hepatocyte apoptosis.

As used herein, the term “acute liver failure” (ALF) is defined as “the rapid development of hepatocellular dysfunction, specifically coagulopathy and mental status changes (encephalopathy) in a patient without known prior liver disease”.

As used herein, the term “chronic liver failure (CLF) usually occurs in the context of cirrhosis, itself potentially the result of many possible causes, such as excessive alcohol intake, hepatitis B or C, autoimmune, hereditary and metabolic causes (such as iron or copper overload, steatohepatitis or non-alcoholic fatty liver disease).”

As used herein, the term “acute on chronic liver failure” refers to the situation where one with chronic liver disease develops features of liver failure. A number of underlying causes may precipitate this, such as alcohol misuse or infection. People with ACLF can be critically ill and require intensive care treatment. and occasionally a liver transplant. Mortality with treatment is 50%.

The present invention provides a therapeutic approach for treating liver diseases, so as to slow or reverse the patient's progression to liver failure. In this context, “liver disease” denotes a condition where liver damage and inflammation threatens to progress to a fatal loss of liver function and/or regenerative capacity. Thus, “liver disease” as used in this description encompasses hepatitis, where inflammation causes damage to liver cells and liver function, whether caused by any virus (viral hepatitis), by a liver toxin (e.g., alcoholic hepatitis), or by autoimmunity (autoimmune hepatitis). Also illustrative of “liver disease” in this description are (A) fatty liver disease (hepatic stetosis), a condition where large vacuoles of triglyceride fat accumulate in liver cells, and (B) non-alcoholic fatty liver disease, which subsumes a spectrum of disease associated with obesity and metabolic syndrome, where either (A) or (B) threatens liver damage so severe as to cause a fatal loss of liver function and/or regenerative capacity.

During a liver disease, damage of the normal liver structure impacts blood supply to the liver. Although up to date, ischemia and hypoxia are not considered as a typical characteristic of liver disease, the inventor discovers that liver injury causes local ischemia and hypoxia of liver. Moreover, another main provoking factor of liver injury is hepatic inflammatory response, resulting in the reduced physiological function and regeneration capacity of the liver. Based on the above pathological characteristics of hypoxia and inflammatory response in a liver disease, the present invention provides a new therapeutic approach for treating liver diseases. The positive and beneficial results: as shown in clinical research and trial results, when administered to patients suffering from liver diseases and conditions, TMZ is capable of providing significant effects in maintaining liver functions and decreasing transaminase levels (including alanine aminotransferase, aspartate transaminase, etc.), with such features as shortening the course of disease, reducing the mortality rate, easy and safe use, and low treatment cost. As TMZ is mainly eliminated unchanged by way of urine, with the elimination half-life of approximately 6 hours and without hepatic metabolism, it causes minimal liver toxicity, while having significant hepatoprotective effect.

In a clinical trial and research involving 165 cases of liver failure by Yu as described in PCT/CN2016/080219, TMZ tablets (20 mg/tablet) were administered orally to the hospitalized liver failure patients, one tablet each time, three times a day, over a 4-week course of treatment. As shown in the clinical trial result, compared with traditional therapeutic approach, the 30-day immortality rate of liver failure patients in the TMZ treatment group dropped from 44% to approximately 18%, and the 90-day immortality rate from 68% to 35%. This suggests that early TMZ intervention in liver failure patients can significantly increase the survival rate and improve their prognosis. Furthermore, the laboratory test indicators suggest that TMZ can significantly raise the decrease rate of alanine aminotransferase and aspartate transaminase. One patient received a PET-CT test of the imaging changes of liver, and the result suggests that, after taking TMZ, the hepatic glucose metabolism of the liver failure patient was remarkably improved. This clinical trial result suggests that early use of TMZ for liver failure patients can significantly improve their liver function, inhibit hepatic inflammatory response, enhance their hepatic glucose metabolism and energy utilization, and improve their chances of survival.

### Formulations

Carriers, excipients and other additives commonly used for pharmaceutical preparations may be used to prepare pharmaceutical compositions comprising trimetazidine or pharmaceutically acceptable salts thereof as active ingredients.

The pharmaceutical composition can be formulated into any suitable formulations. For example, the administration forms may be oral dosage forms, such as tablets, pills, capsules, granules, powders, emulsions, syrups, suspensions, liquid preparations, or non-oral dosage forms, such as intravenous injection or intramuscular injection, suppository, subcutaneous agent, transdermal agent, nasal agent, inhalation. Symptoms, age, gender, etc. of the individual patient should be considered in order to properly determine the dose of a compound.

In the present invention, solid compositions for oral administration may be tablets, powders, granules and the like. In such solid compositions, one or more active substances are mixed with at least one inert excipient (e.g., lactose, mannitol, glucose, hydroxypropylcellulose, microcrystalline cellulose, starch, polyvinyl pyrrolidone, magnesium aluminum silicate, etc.). According to a conventional method, the composition may also contain inert additives such as lubricants (e.g. magnesium stearate), disintegrating agents (e.g., sodium carboxymethyl starch) and dissolution aids. If necessary, tablets or pills may be coated with sugar coating or a gastric or enteric coating agent.

The liquid compositions for oral administration include pharmaceutically acceptable emulsions, solutions, suspensions, syrups, elixirs, and commonly used inert diluent (e.g., purified water, ethanol). In addition to the inert diluent, the composition may also contain additives such as solubilizing agents, wetting agents and suspending agents, as well as sweetener, corrigent, flavoring agents and preservatives.

Injections for non-oral administration include sterile aqueous or non-aqueous liquid preparations, suspensions and emulsions. Diluent for aqueous solution can include (for example) distilled water for injection and physiological saline. Diluent for non-aqueous solution can include (e.g.) propylene glycol, polyethylene glycol, vegetable oils (such as olive oil), alcohols (e.g. ethanol) and polysorbate 80. Such compositions may further contain isotonic agents, preservatives, wetting agents, emulsifying agents, dispersing agents, stabilizing agents, dissolving aids and the like additives. Such compositions may be sterilized by filtration through a bacteria retaining filter, adding bactericides or irradiation with light. In addition, these compositions may be made into sterile solid compositions, and then dissolved or suspended, prior to use, with sterile water or a sterile solvent for injection.

Transmucosal agents, such as inhalations and nasal agents and the like, can be in a solid, liquid, or semi-solid state of use, and can be prepared in accordance with conventionally known methods. For example, an excipient (e.g., lactose and starch), pH adjusting agent, a preservative, surfactants, lubricants, stabilizing agents, thickening agents and the like may be added as needed. You can use a suitable inhalation or insufflation device for administration. For example, a metered dose inhaler or other conventionally known devices or sprayers may be used to administer the compound alone or as a powder mixture after formulation. In addition, the compound may also be combined with a pharmaceutically acceptable carrier, and administered as a solution or suspension. The dry powder inhaler or the like may be used for a single dose or multiple doses, and can use a dry powder or a powder-containing capsule. Further, a pressurized aerosol spray and the like can also be used for administration by the use of a suitable propellant (e.g., chlorofluoroalkane, hydrofluoroalkane, or a suitable gas such as carbon dioxide).

### Dosage Regimen

Generally speaking, in the case of oral administration, daily dosage for adult patients of the compound is about 0.001 mg/kg to 100 mg/kg, a single dose or divided into 2 to 4 times daily. In the case of intravenous administration according to the patient symptoms, generally speaking, daily dose for adult patients is 0.0001 mg/kg to 10 mg/kg, once to more times daily. Further, in the case of using the inhalant administration, generally speaking, daily dosage for adult patients is 0.0001 mg/kg to 1 mg/kg, once to more times daily.

In one embodiment, an adult patient may take orally a pharmaceutical composition comprising about 30-180 mg trimetazidine or a pharmaceutically acceptable salt thereof as an active ingredient daily, in a single dose or 2 to 4 separate doses, with each dose comprising about 10-60 mg trimetazidine. In one embodiment, an adult patient may take orally a pharmaceutical composition comprising about 60 mg trimetazidine or a pharmaceutically acceptable salt thereof as an active ingredient daily, in 3 separate doses, with each dose comprising about 20 mg trimetazidine.

TMZ has been shown to be effective as a drug for acute liver failure as described in PCT/CN2016/080219, the teaching of which is incorporated therein it its entirety by reference. For the sake of concise description, description of such teaching is omitted herein, but can be readily reproduced and incorporated here without undue burden.

### Burst Release and/or Sustained Release

In some embodiments, a formulation of invention can be made to provide various release profiles of TMZ in a single formulation or dosage regimen. Such release profile can be, for example, a burst release of TMZ to meet the urgent need of TMZ in an acute liver failure patient, followed by a sustained release of TMZ to provide a level of TMZ so as to bring about liver protective effect.

Burst release of TMZ can be achieved, for example, by injection or oral administration of TMZ or a pharmaceutically acceptable salt thereof (e.g., a TMZ solution or discrete TMZ particles, which can be administered as solid powder or suspension); and sustained release can be achieved, for example, by a sustained release formulation including embedding TMZ in a biocompatible polymeric material, admixing TMZ with a biocompatible polymeric material, or encapsulating or microencapsulating TMZ with a polymeric material. Preferably, the biocompatible polymeric material is degradable such that the degradation rate of the polymer provides control of the rate of release of TMZ. The polymeric material can be natural polymeric material, e.g., liposome, or synthetic polymer(s), e.g., a polyester such as polylactic acid.

In some embodiments, a formulation that provides burst release—sustained release of TMZ preferably provides a burst release of TMZ between 30 mg to 150 mg within 30 minutes following administration and a sustained release of TMZ at a rate of 10 mg-30 mg per time unit of 4-6 hours over a period of a day to one month, for example, as long as the total dose of TMZ does not exceed the limit set by the prescribed dosage regimen.

Various techniques of formulation to achieve a desired drug release profile(s) are well documented. For example, Encyclopedia of Controlled Drug Delivery, 2-Volume Set, by Edith Mathiowitz (Editor), John Willey & Sons (1999), which is incorporated herein in its entirety by reference, provides general guidance on formulation of a drug to achieve various desirable release profiles.

### Second Agent

In some embodiments, the composition of invention can include a liver protective drug which is not TMZ. In some embodiments, the liver protective drug is as described above.

In some further embodiments, the second agent can be a pharmaceutically or physiologically beneficial drug or agent that improves the overall condition of a subject. In this regard, such second agent can be, for example, one that improves the immune system of the subject, one that improves the neuro or psychological state of the subject, or one that improves the condition of the CNS of the subject.

The foregoing embodiments are provided only for illustration of the preferred embodiments of the present invention; however, the present invention is not limited to the embodiments described above. In the scope of knowledge of a person having ordinary skill in the art, any modification, equivalent substitution, and improvement without departing from the spirit and principles of the present invention, should be considered within the protection scope of the present application.

1. A method of treating acute on chronical liver failure (ACLF) or a related disease in a subject in need thereof, comprising administering to the subject a composition comprising an effective amount of trimetazidine hydrochloride (1-[2,3,4-trimethoxybenzyl] piperazine dihydrochloride) (“Trimetazidine” or “TMZ”) or a pharmaceutically acceptable salt thereof according to a dosage regimen.

2. The method according to claim 1, wherein the effective amount of TMZ is administered to the subject according to a dosage regiment that comprises a dosage of 20-500 mg every 4-12 hours.

3. The method according to claim 1, wherein the dosage regimen comprises a dosage of 20-500 mg every 4-12 hours for a course from 1 day to 2 weeks.

4. The method according to claim 1, wherein the dosage regimen comprises a dosage of 20-500 mg every 4-12 hours for a course from 1 day to 1 month.

5. The method according to claim 1, wherein administering comprises oral administration.

6. The method according to claim 1, wherein administering comprises parenteral injection.

7. The method according to claim 1, wherein administering comprises IV administration.

8. The method according to claim 1, wherein the composition further comprises a pharmaceutically acceptable carrier.

9. The method according to claim 1, wherein the pharmaceutical composition reduces activation of resting T lymphocytes to activated lymphocytes in the subject.

10. The method according to claim 1, wherein the pharmaceutical composition reduces fatty acid metabolism in hepatocytes and promotes aerobic oxidation of glucose in the subject.

11. The method according to claim 1, wherein the pharmaceutical composition reduces beta-oxidation in hepatocytes and reduces formation of ROS in the subject.

12. The method according to claim 1, wherein the pharmaceutical composition reduces blood alanine aminotransferase (ALT) level in the subject.

13. The method according to claim 1, wherein the pharmaceutical composition reduces blood aspartate transaminase (AST) level in the subject.

14. The method according to claim 1, wherein the related disorder does not include angina pectoris, coronary insufficiency, previous myocardial infarction, coronary heart disease, vertigo or tinnitus.

15. The method according to claim 1, wherein the related disorder is selected from the group consisting of hepatopathy (liver damage without major inflammation), hepatitis (with inflammation) and cirrhosis (structure damage may or may not have liver failure).

16. The method according to claim 1, wherein the related disorder is selected from the group consisting of viral hepatitis, non-specific virus hepatitis, drug or medication induced liver injury or damage, toxin induced hepatitis such as herbs or mushroom induced hepatitis or hepatopathy, alcoholic hepatitis, autoimmune hepatitis, alcoholic fatty liver disease, or non alcoholic fatty liver disease(NAFLD), non-alcoholic steatohepatitis (NASH), metabolic or genetically related liver diseases (Hemochromatosis, Wilson's diseases), ischemic liver injury (shock live), sepsis (bacteria or other microorganism infection) induced liver failure, and congestive hepatopathy (heart failure induced liver failure).

17. The method according to claim 1, wherein the subject is a human being or an animal.

18. The method according to claim 1, wherein the composition comprises a second agent.

19. A formulation, comprising trimetazidine hydrochloride (1-[2,3,4-trimethoxybenzyl] piperazine dihydrochloride) (“Trimetazidine” or “TMZ”) or a pharmaceutically acceptable salt thereof and a carrier, the formulation provides a release profile of TMZ or a pharmaceutically acceptable salt thereof, which profile comprising a burst release of the TMZ or a pharmaceutically acceptable salt thereof followed by a sustained release of TMZ or a pharmaceutically acceptable salt thereof.

20. The formulation according to claim 19, wherein the burst release releases 30 mg to 180 mg of TMZ or a pharmaceutically acceptable salt thereof following administration of the TMZ or a pharmaceutically acceptable salt thereof to a patient.

21. The formulation according to claim 19, wherein the sustained release releases 10 mg to 30 mg of TMZ or a pharmaceutically acceptable salt thereof every 4 to 6 hours over a period from one day to one month.

22. The formulation according to claim 19, wherein the sustained release of TMZ or a pharmaceutically acceptable salt thereof is provided by embedding the TMZ or the pharmaceutically acceptable salt thereof in a biocompatible polymeric material, admixing TMZ or the pharmaceutically acceptable salt therof with a biocompatible polymeric material, or encapsulating or microencapsulating TMZ or the pharmaceutically acceptable salt thereof with a polymeric material.

23. The formulation according to claim 19, which is an oral formulation or an injection formulation.

24. A method of making a formulation, comprising combining an effective amount of trimetazidine (1-[2,3,4-trimethoxybenzyl] piperazine) dihydrochloride (“Trimetazidine” or “TMZ”) for acute on chronical liver failure (ACLF) or a related disease in a subject in need thereof and a carrier; and
forming the formulation,
wherein the formulation is structured to provide a release profile of TMZ, which profile comprising a burst release of the TMZ followed by a sustained release of TMZ.

25. The method according to claim 24, wherein the formulation is structured to provide a burst release release of 30 mg to 180 mg of TMZ following administration of the TMZ to a patient.

26. The method according to claim 24, wherein the formulation is structured to provide a sustained release release of 10 mg to 30 mg of TMZ every 4 to 6 hours over a period from one day to one month.

27. A method of treating a liver disorder or a related disease, comprising administering to a patient in need thereof a formulation, the formulation comprising trimetazidine hydrochloride (1-[2,3,4-trimethoxybenzyl] piperazine dihydrochloride) (“Trimetazidine” or “TMZ”) or a pharmaceutically acceptable salt thereof and a carrier, the formulation provides a release profile of TMZ or a pharmaceutically acceptable salt thereof, which profile comprising a burst release of the TMZ or a pharmaceutically acceptable salt thereof followed by a sustained release of TMZ or a pharmaceutically acceptable salt thereof.

28. The method according to claim 27, wherein the burst releases 30 mg to 180 mg of TMZ or a pharmaceutically acceptable salt thereof following administration of the TMZ or a pharmaceutically acceptable salt thereof to a patient.

29. The method according to claim 27, wherein the sustained release releases 10 mg to 30 mg of TMZ or a pharmaceutically acceptable salt thereof every 4 to 6 hours over a period from one day to one month.

30. The method of claim 27, wherein the liver disorder is acute on chronic liver failure.
